(19) United States
(12) Patent Application Publication
(10) Pub. No.: US 2025/0107911 A1
(43) Pub. Date: Apr. 3, 2025

(54) PROSTHETIC VALVE CRIMPING DEVICES

(71) Applicant: Anteris Technologies Corporation, Eagan, MN (US)

(72) Inventor: Alford Lee McLevish, Roseville, MN (US)

(21) Appl. No.: 18/901,345

(22) Filed: Sep. 30, 2024

Related U.S. Application Data
(60) Provisional application No. 63/587,337, filed on Oct. 2, 2023.

(51) Int. Cl. A61F 2/95

(52) U.S. Cl. CPC A61F 2/9524

(57) ABSTRACT

Devices can be used to radially crimp medical devices to a smaller diameter in an accurate and controllable manner. For example, this document describes crimping devices for radially crimping prosthetic heart valves on to a balloon catheter in a precise and controllable manner. The prosthetic heart valve crimping devices described herein are designed to be efficiently manufacturable and operable in a user-friendly manner. In some examples, a two-stage crimping process, including the use of a crimping accessory for the second stage, is utilized to radially crimp a localized portion of the medical devices to a smaller diameter in an accurate and controllable manner.

### CROSS-REFERENCE TO RELATED APPLICATIONS

This application claims the benefit of U.S. Provisional Application Ser. No. 63/587,337 filed Oct. 2, 2023. The disclosure of the prior application is considered part of (and is incorporated by reference in) the disclosure of this application.

### BACKGROUND

### 1. Technical Field

This document relates to devices for radially crimping medical devices and methods for their use. For example, this document relates to devices for radially crimping prosthetic heart valves on to a balloon catheter in a precise and controllable manner.

### 2. Background Information

Some prosthetic heart valves are deployable to a native heart valve location using a system of catheters that carry the prosthetic heart valve while it is in a low profile (crimped) configuration. Most available transcatheter prosthetic heart valves are designed to be either balloon-expandable or self-expanding.

Prior to deployment, balloon-expandable transcatheter prosthetic heart valves are typically radially crimped onto a balloon and then loaded into a sheath in preparation for deployment.

Crimping tools are designed to crimp or reduce the diameter of balloon-expandable transcatheter prosthetic heart valves onto a balloon.

### SUMMARY

This document describes devices for radially crimping medical devices and methods for their use. For example, this document describes crimping devices for radially crimping prosthetic heart valves on to a balloon catheter in a precise and controllable manner. The prosthetic heart valve crimping devices described herein are designed to be efficiently manufacturable and operable in a user-friendly manner.

In one aspect, this disclosure is directed to a prosthetic valve crimping device includes: (i) a base member; (ii) a slotted plate affixed to the base member, the slotted plate defining a center and a plurality of slots extending radially from the center; (iii) a housing rotatably coupled to the slotted plate, the housing defining a plurality of arcuate cam slots; and (iv) a plurality of crimping jaws slidably engaged in the plurality of slots. Each crimping jaw comprises one or more posts slidably disposed in one or more arcuate cam slots of the plurality of arcuate cam slots, and one or more flexible tabs by which the crimping jaw is snapped into engagement in a slot of the plurality of slots.

Such a prosthetic valve crimping device may optionally include one or more of the following features. Each slot of the plurality of slots may be linear. The one or more posts may comprise two posts. Each arcuate cam slot of the plurality of arcuate cam slots may have a single post of the one or more posts slidably disposed therein. The housing may include two housing portions. Each housing portion may define multiple arcuate cam slots of the plurality of arcuate cam slots. The housing may include a handle for a user to rotate the housing relative to the base member. The prosthetic valve crimping device may also include a rotational limiter pivotably coupled to the base member. The rotational limiter may be pivotable between: (i) a first position in which the rotational limiter is spaced apart from the housing and (ii) a second position in which the rotational limiter is engaged with the housing. The housing may define an open center area. An end portion of each crimping jaw may be positioned in the open center area. In some embodiments, the end portion of each crimping jaw is wider than the housing. In some embodiments, the one or more flexible tabs consists of four flexible tabs.

This disclosure is also directed to a method of assembling a prosthetic valve crimping device. The method may include snapping each crimping jaw of the plurality of crimping jaws into slidable engagement with a respective slot of the plurality of slots by flexing the one or more flexible tabs thereof. The method may also include snapping the slotted plate into engagement with the base member. The method may also include snapping two housing portions of the housing into engagement with the slotted plate. In some embodiments, zero fasteners are used to assemble an entirety of the prosthetic valve crimping device.

This disclosure is also directed to a prosthetic valve crimping device that includes: (a) a base member; (b) a housing that is rotatable relative to the base member, the housing defining a center and a central axis, wherein the housing has a width measured along the central axis; and (c) a plurality of crimping jaws radially movable relative to the housing, and end portion of each crimping jaw having a width measured along the central axis. The width of the end portion of each crimping jaw is wider than the width of the housing.

Such a prosthetic valve crimping device may optionally include one or more of the following features. The prosthetic valve crimping device may also include a slotted plate affixed to the base member. The slotted plate may define a plurality of slots extending radially from the center. In some embodiments, the plurality of crimping jaws are slidably disposed in the plurality of slots. The housing may comprise two housing portions. Each housing portion may define multiple arcuate cam slots of the plurality of arcuate cam slots. Each crimping jaw may include two posts. In some embodiments, each arcuate cam slot of the plurality of arcuate cam slots has a single post of the plurality of crimping jaws slidably disposed therein.

Particular embodiments of the subject matter described in this document can be implemented to realize one or more of the following advantages. In some embodiments, the prosthetic heart valve crimping devices described herein are designed to be efficiently manufactured. For example, in some embodiments the components of the crimping devices can be snapped together without using mechanical fasteners. Secondly, in some embodiments the crimping jaws of the prosthetic heart valve crimping devices are wider than the housing of the devices. Accordingly, a user of the crimping device is able to readily visualize the crimping jaws, the prosthetic heart valve, and the balloon so that the crimping can be performed in a precise manner. In some embodiments, various heart conditions can be treated in a minimally invasive fashion as a result of using the crimping and devices and methods provided herein. Such minimally invasive techniques can reduce recovery times, patient discomfort, and treatment costs.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a perspective view of an example prosthetic valve crimping device in accordance with some embodiments provided herein.

FIG. 2 is a perspective view of the prosthetic valve crimping device of FIG. 1 in another configuration.

FIG. 3 is a side view of the prosthetic valve crimping device of FIG. 1.

FIG. 4 is a perspective view of the prosthetic valve crimping device of FIG. 1 in another configuration.

FIG. 5 is an exploded view of the prosthetic valve crimping device of FIG. 1.

FIG. 6 is a perspective view of a slotted plate component of the prosthetic valve crimping device of FIG. 1.

FIG. 7 is a cross-section view of a lower portion of the prosthetic valve crimping device of FIG. 1.

FIG. 8 is a bottom view of the prosthetic valve crimping device of FIG. 1.

FIGS. 9 and 10 are perspective views of a crimping jaw component of the prosthetic valve crimping device of FIG. 1.

FIG. 11 is a side view of a sub assembly of the prosthetic valve crimping device of FIG. 1 that includes the slotted plate and multiple crimping jaws.

FIG. 12 is a perspective view of an example crimping accessory device in accordance with some embodiments.

FIG. 13 is a cross-section view of the crimping accessory of FIG. 12 in use within the prosthetic valve crimping device of FIG. 1.

FIG. 14 is an expanded view of a portion of FIG. 13.

FIG. 15 is a longitudinal cross-sectional view of another example crimping accessory device in accordance with some embodiments.

Like reference numbers represent corresponding parts throughout.

### DETAILED DESCRIPTION

This document describes devices for radially crimping medical devices and methods for their use. For example, this document describes crimping devices for radially crimping prosthetic heart valves on to a balloon catheter in a precise and controllable manner. The prosthetic heart valve crimping devices described herein are designed to be efficiently manufacturable and operable in a user-friendly manner.

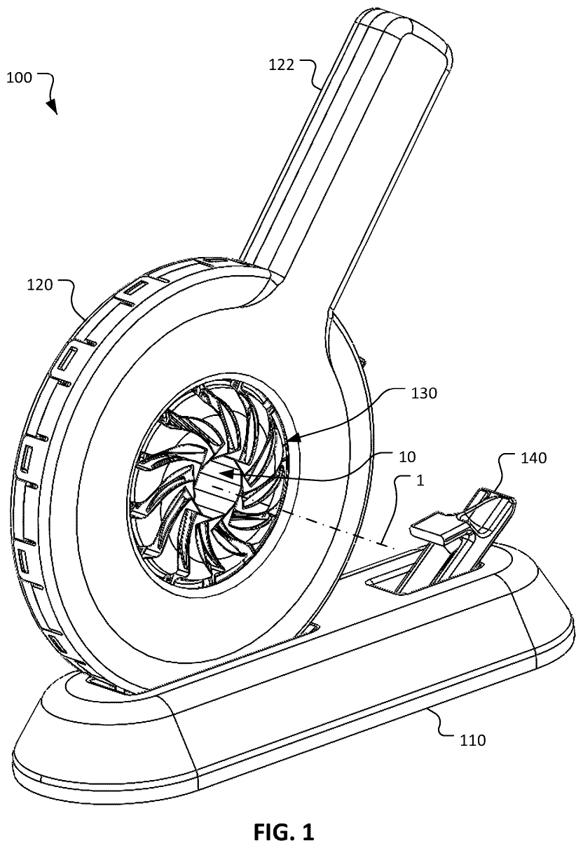

FIG. 1 illustrates an example crimping device 100. The crimping device 100 can be used to radially crimp various types of medical devices. In one such example, the crimping device 100 can be used to radially crimp a balloon-expandable prosthetic heart valve onto a balloon of a delivery catheter. Accordingly, in some embodiments the crimping device 100 can be referred to as a prosthetic heart valve crimping device 100.

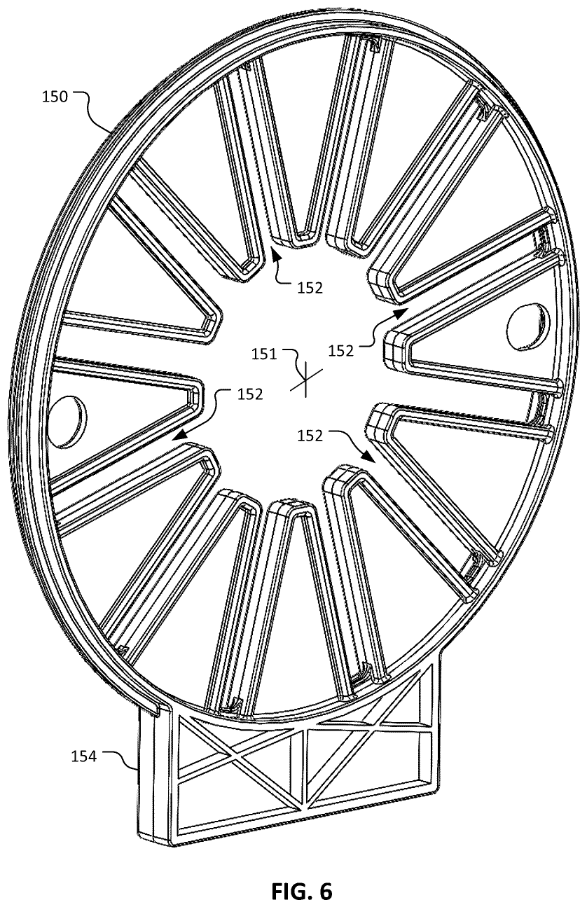

The depicted embodiment of the crimping device 100 includes a base member 110, a housing 120, a plurality of crimping jaws 130, a rotational limiter 140, and a slotted plate 150 (e.g., see FIG. 6). The housing 120 and the plurality of crimping jaws 130 define a center opening 10 (or iris 10) with a center axis 1.

The center opening 10 is where the medical device (e.g., prosthetic heart valve) is positioned when the crimping of the medical device is preparing to take place. That is, and as described further below, when a medical device is positioned in the center opening 10 and the housing 120 is then rotated, the plurality of crimping jaws 130 move radially inward toward the center axis 1 and the radial crimping of the medical device takes place.

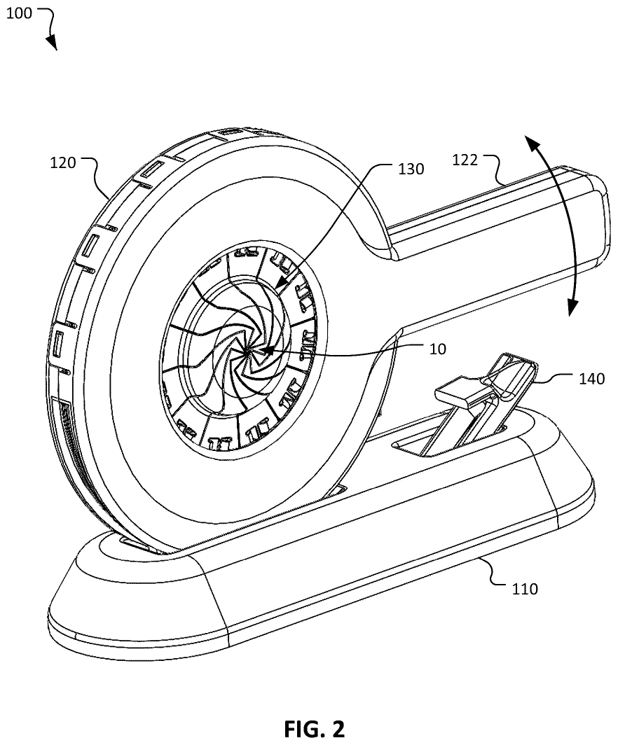

The housing 120 is rotatable relative to the base member 110. In the depicted embodiment, the housing 120 includes a handle 122 by which a user can readily rotate the housing 120 relative to the base member 110. The rotation of the housing 120 can be visualized, for example, by comparing FIG. 1 to FIG. 2.

The rotation of the housing 120 causes the crimping jaws 130 to move radially inward or outward. The radial movement of the crimping jaws 130 can also be visualized, for example, by comparing FIG. 1 to FIG. 2. In FIG. 2, the center opening 10 is much smaller in diameter than the center opening 10 is in FIG. 1.

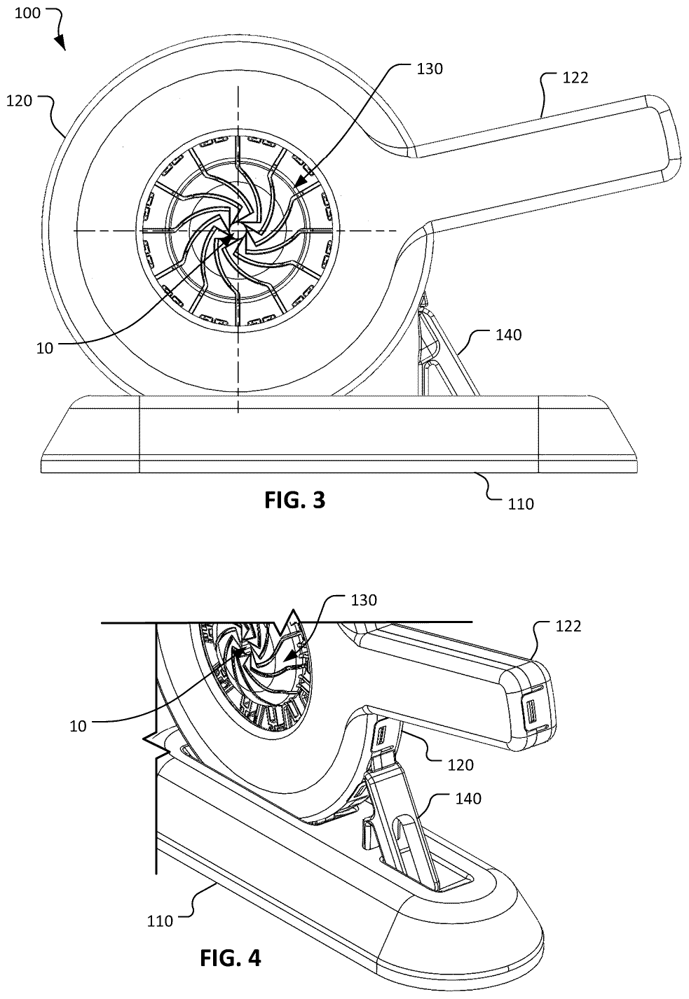

The crimping device 100 also includes the rotational limiter 140. The rotational limiter 140 is pivotably coupled to the base member 110. As a result, the rotational limiter 140 is pivotable between: (i) a first position in which the rotational limiter 140 is spaced apart from the housing 120 as shown in FIGS. 1 and 2 and (ii) a second position in which the rotational limiter 140 is engaged with the housing 120 as shown in FIGS. 3 and 4.

When the rotational limiter 140 is in the first position, the housing 120 is freely rotatable without any limitations from the rotational limiter 140. However, when the rotational limiter 140 is in the second position (as shown in FIGS. 3 and 4), the housing 120 can be rotated only to a particular position/extent. Said another way, when the rotational limiter 140 is in the second position, the extent of the radial compression from the crimping jaws 130 is limited. That is, the opening 10 can be made smaller only to a certain size of opening 10 (e.g., about 9 mm+/−2 mm in some embodiments). However, when the rotational limiter 140 is in the first position, the opening 10 can be made even smaller (e.g., about 2.5 mm+/−1 mm).

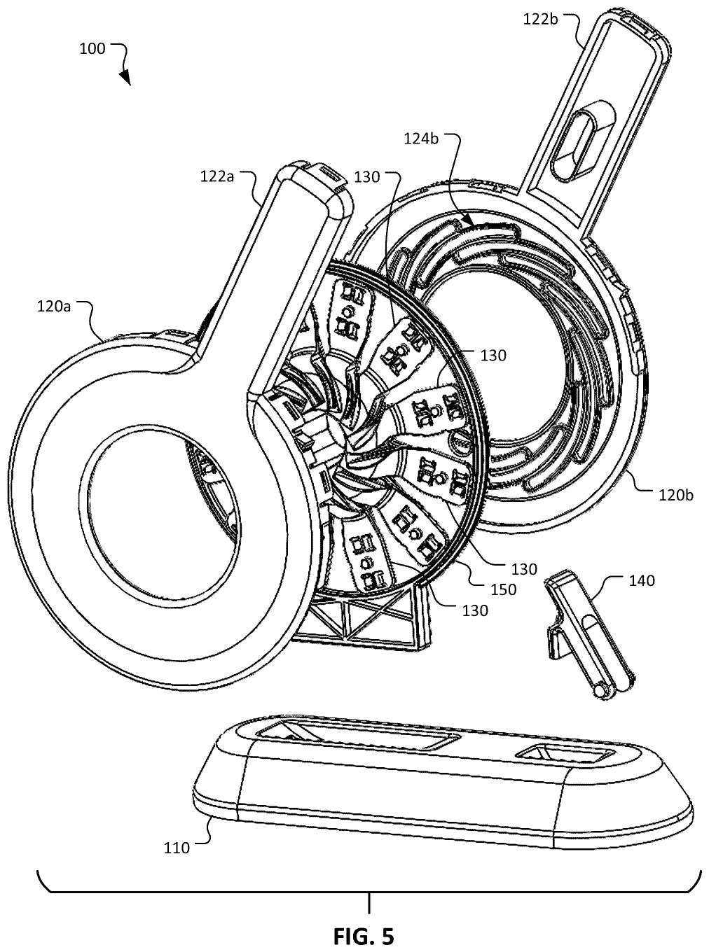

Referring to FIG. 5, in this exploded view it can be seen that the housing 120 is made of two housing portions 120a and 120b.

The two housing portions 120a/b are made to snap into engagement with each other. That is, the housing portions 120a/b include mechanical features such as, but not limited to, flexible tabs that define openings that engage with protrusions on the opposite housing portion. Accordingly, no conventional fasteners (e.g., screws, rivets, clips, etc.) are needed to engage the two housing portions 120a/b together. This makes assembly of the two housing portions 120a/b very efficient and low cost.

Each of the housing portions 120a/b defines a plurality of actuate cam slots 124a (not visible) and 124b. As described further below, posts of the crimping jaws 130 are slidably engaged in the actuate cam slots 124a/b. In result, as the housing 120 is rotated by a user, the actuate cam slots 124a/b drive the crimping jaws 130 radially inward or outward (depending on the direction of rotation of the housing 120 relative to the base member 110).

Still referring to FIG. 5, the slotted plate 150 and the crimping jaws 130 are positioned inside of the two housing portions 120a/b. As described further below, the slotted plate 150 is affixed to the base member 110. The housing portions 120a/b are, in turn, rotatably coupled to the slotted plate 150. Accordingly, the housing 120 is rotatable relative to the base member 110 and to the slotted plate 150.

Referring also to FIG. 6, the slotted plate 150 defines a plurality of slots 152 extending radially from the center 151 of the slotted plate 150. In the depicted embodiment, the slots 152 are linear.

The crimping jaws 130 are slidably coupled in the slots 152. In particular, a single jaw of the plurality of crimping jaws 130 is slidably engaged in a single, corresponding slot of the plurality of slots 152, in a one-to-one manner. In some embodiments, a lubricant (e.g., silicone grease, graphite, or other types of lubricants) is applied to the inner walls of the slots 152 to reduce the sliding friction between the slots 152 and the crimping jaws 130. Such lubricant can reduce the amount of user-generated force needed to crimp a prosthetic heart valve as described herein.

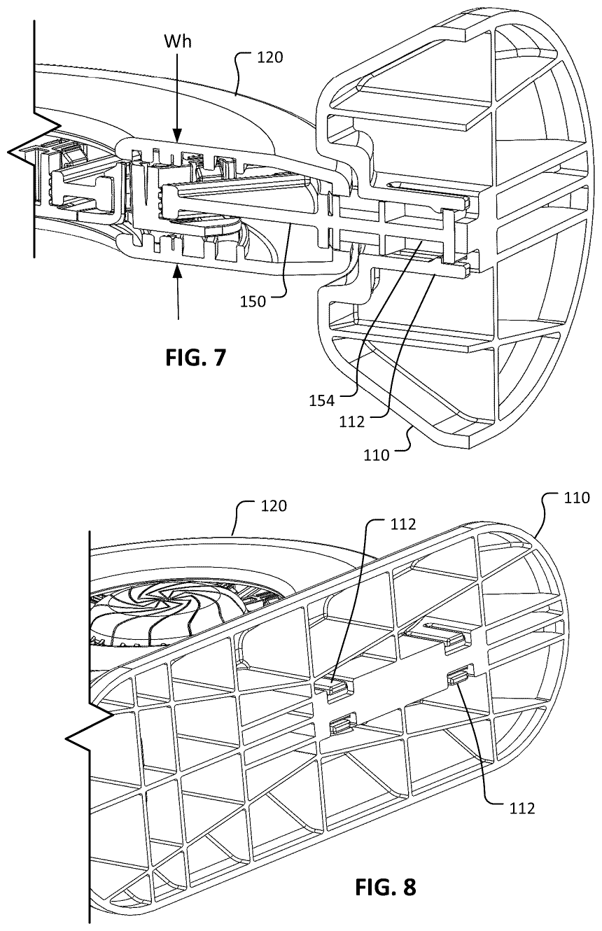

The slotted plate 150 also includes a flange 154. The flange 154 is used to affix the slotted plate 150 to the base member 110. Specifically, and as shown in FIGS. 7 and 8, the flange 154 is snapped into an affixed engagement with the base member 110. In the depicted embodiment, the base member 110 includes flexible tabs 112 that the flange 154 snaps into engagement with. Accordingly, no conventional fasteners (e.g., screws, rivets, clips, etc.) are needed to affix the slotted plate 150 to the base member 110. This makes the assembly of the two components very efficient and low cost.

FIG. 7 shows that the housing 120 has a width Wh that is measured along the center axis 1 (FIG. 1).

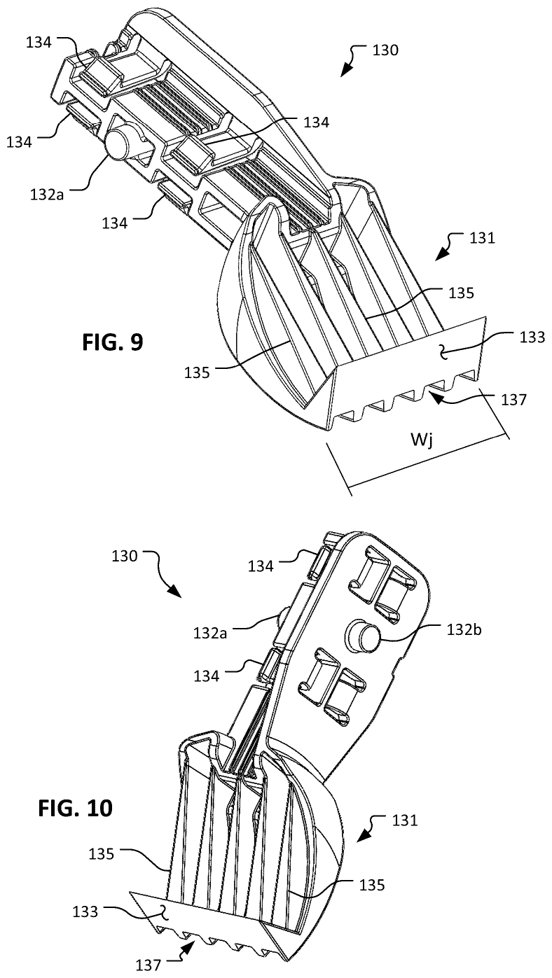

FIGS. 9 and 10 show a single crimping jaw 130 of the plurality of crimping jaws 130 in isolation so that its features are more readily visible. In the depicted embodiment, the crimping jaw 130 is a unitary construct (e.g., injection molded, in some embodiments).

The end portion 131 of the crimping jaw 130 that performs the radial compression has a face surface 133 and a width Wj that is measured along the center axis 1 (FIG. 1). The width Wj is greater than the width Wh of the housing 120 (FIG. 7). This is an advantageous arrangement because when a user is aligning a prosthetic valve (or other medical device) relative to the crimping jaws 130, the housing 120 is not a visual impediment.

In some embodiments, the width Wj is within a range of 30 mm to 40 mm, or 34 mm to 44 mm, or 38 mm to 42 mm, or 40 mm to 44 mm, without limitation.

The crimping jaw 130 includes one or more posts (the crimping jaw 130 includes two posts 132a and 132b in the depicted embodiment). The posts 132a/b are each slidably disposed in a respective single arcuate cam slot 124a/b of the plurality of arcuate cam slots 124a/b defined by the housing portions 120a/b (see FIG. 5). The sliding engagement of the posts 132a/b in the arcuate cam slots 124a/b drive/force the crimping jaws 130 to move when the housing 120 is rotated relative to the base member 110. The slots 152 in the slotted plate 150 guide the crimping jaws 130 to move purely radially as the arcuate cam slots 124a/b drive/force the crimping jaws 130 to move in response to the rotational movement of the housing 120.

The face surface 133 is structurally reinforced by one or more gussets 135 that extend from a rear side of the face surface 133 to other portions of the end portion 131 of the crimping jaw 130. The one or more gussets 135 add strength and rigidity to the end portion 131 of the crimping jaw 130 when the face surface 133 is radially compressing a prosthetic valve (or other medical device).

The end portion 131 of the crimping jaw 130 also defines one or more grooves 137. The one or more grooves 137 slidably receive the one or more gussets 135 of an adjacent crimping jaw 130. Accordingly, each crimping jaw 130 is reinforced by an adjacent crimping jaw 130. The nesting effect between the one or more gussets 135 and the one or more grooves 137 also provides axial strength to the crimping jaws 130. That is, a single crimping jaw 130 cannot be made to deflect axially (along the axis 1; FIG. 1) unless all of the crimping jaws 130 are made to deflect axially. This is the case because all of the crimping jaws 130 have the one or more gussets 135 that are nested in the one or more grooves 137 of an adjacent crimping jaw 130.

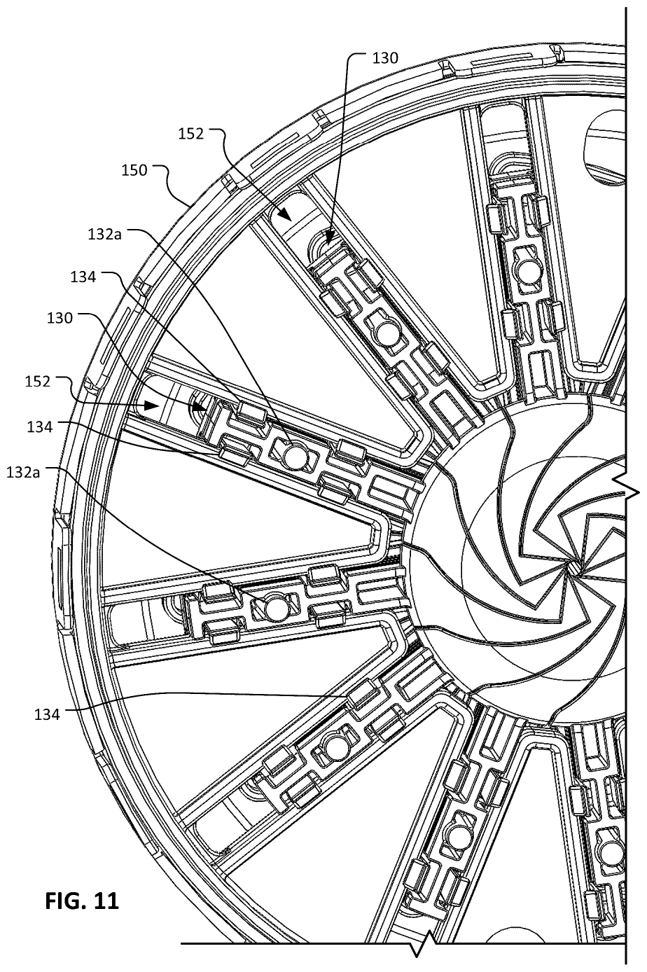

Also referring to FIG. 11, the crimping jaw 130 also includes one or more flexible tabs (the crimping jaw 130 includes four flexible tabs 134 in the depicted embodiment). The flexible tabs 134 are used to snap the crimping jaws 130 into engagement with the slots 152 in the slotted plate 150. Accordingly, no conventional fasteners (e.g., screws, rivets, clips, etc.) are needed to engage the crimping jaws 130 with the slotted plate 150. This makes assembly of the crimping jaws 130 with the slotted plate 150 very efficient and low cost.

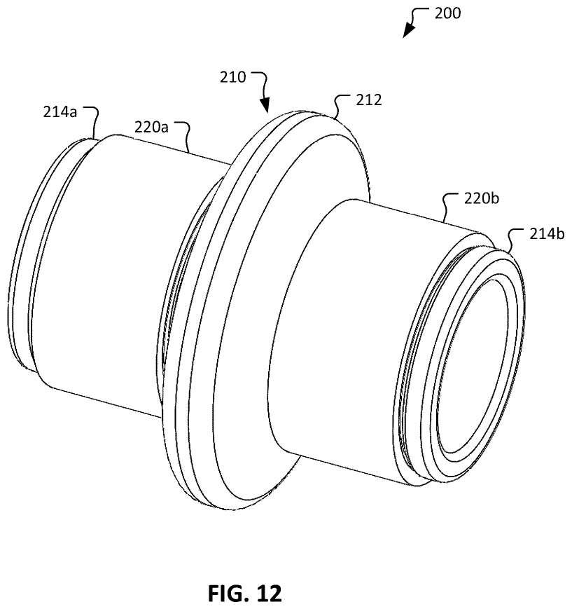

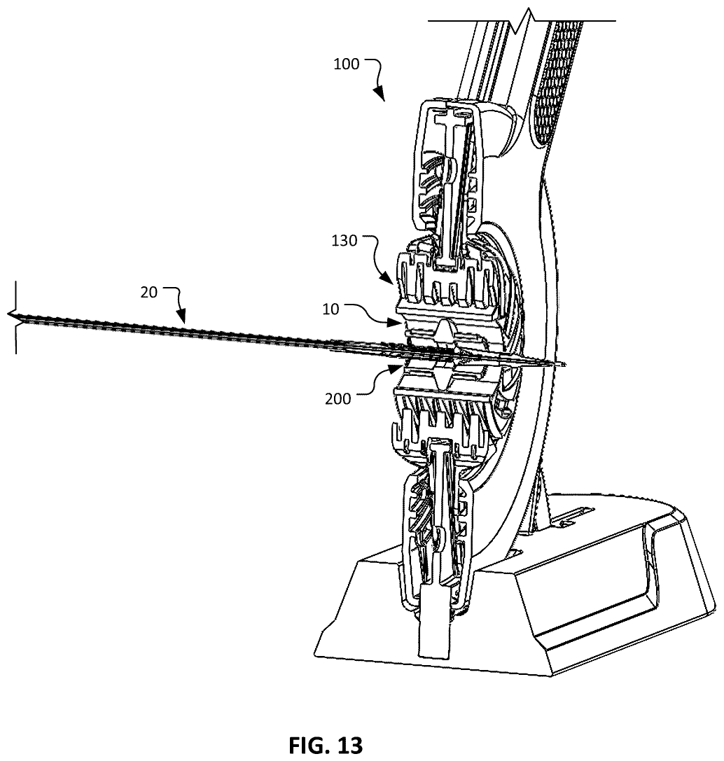

Referring to FIGS. 12-14, in some cases a crimping accessory device 200 (or more simply “crimping accessory 200”) can be used to engender or apply additional radial crimping to a portion of a balloon-expandable prosthetic heart valve 40 onto a balloon of a delivery catheter system 20. That is, by using the crimping accessory 200 as described further below, a particular portion of a prosthetic heart valve 40 can be radially crimped to a smaller diameter than the other remaining portions of the prosthetic heart valve 40.

In the depicted embodiment, the crimping accessory 200 includes a flexible sleeve 210, a first rigid collar 220a, and a second rigid collar 220b.

The flexible sleeve 210 has a middle portion 212, a first tubular member 214a, and a second tubular member 214b. The first tubular member 214a extends from the middle portion 212, and the second tubular member 214b extends from the middle portion 212 in the direction opposite of the first tubular member 214a. In other words, the middle portion 212 is located between the first and second tubular members 214a-b.

The first and second rigid collars 220a-b are mounted on/over the first and second tubular members 214a-b respectively. The first and second rigid collars 220a-b are open cylindrical members through which the first and second tubular members 214a-b extend.

The flexible sleeve 210 is made of a compliant material such as, but not limited to, silicone, thermoplastic elastomer (TPE), neoprene rubber, EPDM rubber, nitrile rubber, and the like. In some embodiments, the flexible sleeve 210 has a durometer in a range of Shore 40A to Shore 60A, without limitation.

The first and second rigid collars 220a-b are made of relatively rigid and inflexible materials such as, but not limited to, PVC, polycarbonate, acrylic, aluminum, and the like.

In some embodiments, one or more of the flexible sleeve 210 and/or the first and second rigid collars 220a-b are made of optically/visually transparent materials. In particular embodiments, the flexible sleeve 210 and the first and second rigid collars 220a-b are all optically/visually transparent. Such optical transparency can help the user to position the middle portion 212 around the region of the prosthetic heart valve 40 that is to be further compressed/crimped using the crimping accessory 200.

The middle portion 212 of the flexible sleeve 210 has a larger outer diameter than the outer diameters of the first and second rigid members 220a-b. Accordingly, as described further below, when the crimping accessory 200 is placed in the center opening 10 (or iris 10) of the crimping device 100 and a crimping force is applied, the portion of the prosthetic heart valve 40 that is positioned within the middle portion 212 will receive additional radial crimping force (while other portions of the prosthetic heart valve will not receive any additional radial crimping force because of the protection provided by the incompressible first and second rigid members 220a-b). In other words, the additional radial crimping force that can be applied using the crimping accessory 200 is localized to the portion of the prosthetic heart valve 40 that is positioned within the inner diameter of the middle portion 212.

In the depicted embodiment of the crimping accessory 200, the middle portion 212 has a smaller inner diameter than the inner diameters of the first and second tubular members 214a-b. This arrangement also helps to ensure that the additional radial crimping force that can be applied using the crimping accessory 200 is localized to the portion of the prosthetic heart valve 40 that is positioned within the inner diameter of the middle portion 212.

The crimping accessory 200 is used as a tool for the second step of a two-step crimping process. In some examples, such a two-step crimping process is performed in the following manner. First, a new (typically), radially expanded balloon-expandable prosthetic heart valve 40 is placed over an uninflated balloon of the delivery catheter system 20. That is, the balloon of the delivery catheter system 20 is positioned within the inner diameter of the radially expanded balloon-expandable prosthetic heart valve 40. Accurate relative longitudinal positioning of the radially expanded balloon-expandable prosthetic heart valve 40 on the balloon of the delivery catheter system 20 is important to ensure that the crimping process will provide a good result.

Second, the radially expanded balloon-expandable prosthetic heart valve 40 with the balloon of the delivery catheter system 20 located therein is placed within the center opening 10 (or iris 10) of the crimping device 100.

Third, the crimping device 100 is activated to radially compress (crimp) the balloon-expandable prosthetic heart valve 40 onto the balloon of the delivery catheter system 20. This is the first stage of crimping.

Fourth, when additional radial compression of a localized portion of the balloon-expandable prosthetic heart valve 40 is desired, the crimping accessory 200 is placed over the crimped balloon-expandable prosthetic heart valve 40 which is crimped on the balloon of the delivery catheter system 20. The middle portion 212 of the crimping accessory 200 is aligned/positioned in the proper longitudinal location relative to crimped balloon-expandable prosthetic heart valve 40 so that the localized portion of the balloon-expandable prosthetic heart valve 40 that is targeted for further radial compression is positioned within the middle portion 212 of the crimping accessory 200.

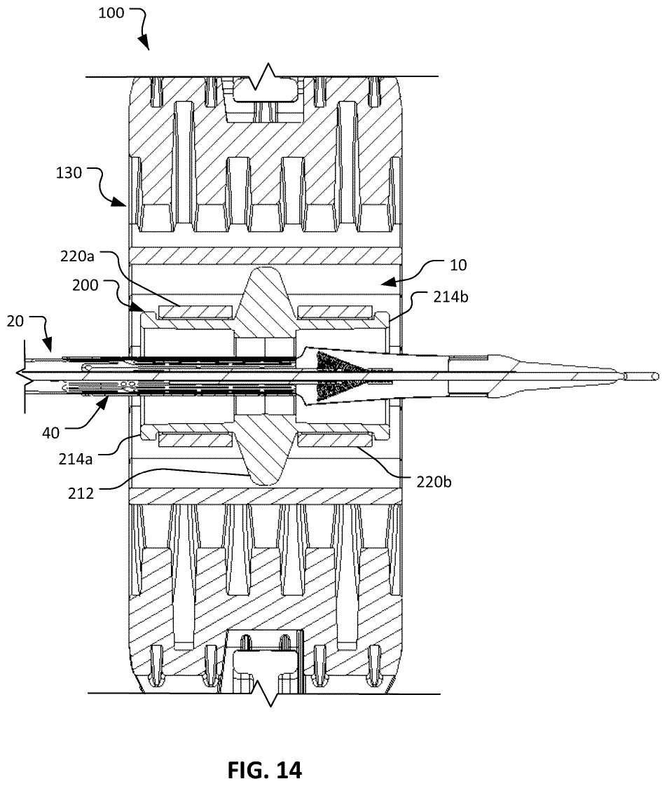

Fifth, the combination of the crimping accessory 200 and the crimped balloon-expandable prosthetic heart valve 40 (which is crimped on the balloon of the delivery catheter system 20) from step four is placed within the center opening 10 (or iris 10) of the crimping device 100. This is illustrated in FIG. 14, for example. In the example depicted in FIG. 14, a distal end portion of the balloon-expandable prosthetic heart valve 40 is targeted for further radial compression (because it is located within the inner diameter of the middle portion 212 of the crimping accessory 200).

Sixth, the crimping device 100 is activated a second time to radially compress (crimp) the crimping accessory 200 so that the middle portion 212 exerts additional radial compression onto the targeted local portion of the balloon-expandable prosthetic heart valve 40. This is the second stage of crimping. In result, the targeted local portion of the balloon-expandable prosthetic heart valve 40, after the second crimping, has a smaller outer diameter than the other portions of the balloon-expandable prosthetic heart valve 40 that do not receive the second stage of crimping.

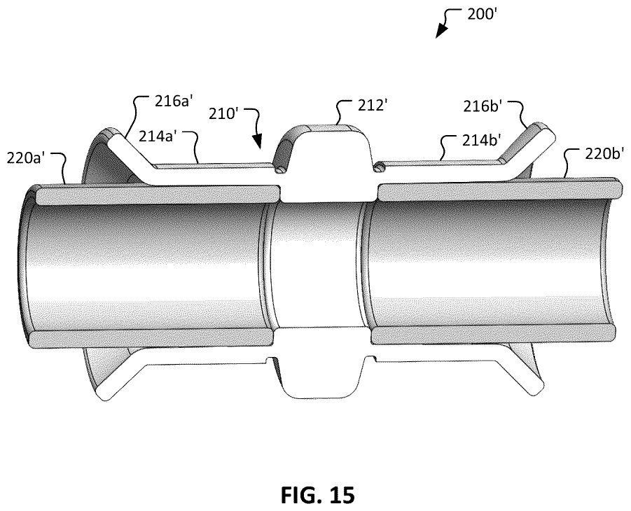

FIG. 15 shows another example crimping accessory device 200′ (or more simply “crimping accessory 200”) can be used to engender or apply additional radial crimping to a portion of a balloon-expandable prosthetic heart valve 40 onto a balloon of a delivery catheter system 20 in the same manner as described above.

In the depicted embodiment, the crimping accessory 200′ includes a flexible sleeve 210′, a first rigid sleeve 220a′, and a second rigid sleeve 220b′.

The flexible sleeve 210′ has a middle portion 212′, a first tubular member 214a′, a second tubular member 214b′, a first flared end portion 216a′, and a second flared end portion 216b′. The first tubular member 214a′ extends from the middle portion 212′, and the second tubular member 214b′ extends from the middle portion 212′ in the direction opposite of the first tubular member 214a′. In other words, the middle portion 212′ is located between the first and second tubular members 214a′-b′.

The first flared end portion 216a′ is attached to, or is contiguous with, the first tubular member 214a′, and the second flared end portion 216b′ is attached to, or is contiguous with, the second tubular member 214b′. The first and second flared end portions 216a′-b′ comprise the extreme opposite end portions of the flexible sleeve 210′. The first and second flared end portions 216a′-b′ are flared to help to establish and maintain the crimping accessory 200′ in a radially central/concentric position within the center opening 10 (or iris 10) of the crimping device 100. The first and second flared end portions 216a′-b′ are radially compressible and can become fully unflared when compressed.

The first and second rigid sleeves 220a′-b′ are mounted inside of the first and second tubular members 214a′-b′ respectively. The first and second rigid collars 220a′-b′ are open cylindrical members over which the first and second tubular members 214a′-b′ extend.

The flexible sleeve 210′ is made of a compliant material such as, but not limited to, silicone, thermoplastic elastomer (TPE), neoprene rubber, EPDM rubber, nitrile rubber, and the like. In some embodiments, the flexible sleeve 210′ has a durometer in a range of Shore 40A to Shore 60A, without limitation.

The first and second rigid sleeves 220a′-b′ are made of relatively rigid and inflexible materials such as, but not limited to, PVC, polycarbonate, acrylic, aluminum, and the like.

In some embodiments, one or more of the flexible sleeve 210′ and/or the first and second rigid sleeves 220a′-b′ are made of optically/visually transparent materials. In particular embodiments, the flexible sleeve 210′ and the first and second rigid sleeves 220a1-b′ are all optically/visually transparent. Such optical transparency can help the user to position the middle portion 212′ around the region of the prosthetic heart valve 40 that is to be further compressed/crimped using the crimping accessory 200′.

The middle portion 212′ of the flexible sleeve 210′ has a larger outer diameter than the outer diameters of the first and second rigid sleeves 220a′-b′. Accordingly, as described further herein, when the crimping accessory 200′ is placed in the center opening 10 (or iris 10) of the crimping device 100 and a crimping force is applied, the portion of the prosthetic heart valve 40 that is positioned radially within the middle portion 212′ will receive additional radial crimping force (while other portions of the prosthetic heart valve 40 will not receive any additional radial crimping force because of the protection provided by the first and second sleeves members 220a′-b′ which are incompressible). In other words, the additional radial crimping force that can be applied using the crimping accessory 200′ is localized to the portion of the prosthetic heart valve 40 that is positioned within the inner diameter of the middle portion 212′.

The crimping accessory 200′ is used as a tool for the second step of a two-step crimping process. In some examples, such a two-step crimping process is performed in the manner described above.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results.

What is claimed is:

1. A prosthetic valve crimping device comprising:
a base member;
a slotted plate affixed to the base member, the slotted plate defining a center and a plurality of slots extending radially from the center;
a housing rotatably coupled to the slotted plate, the housing defining a plurality of arcuate cam slots; and
a plurality of crimping jaws slidably engaged in the plurality of slots, each crimping jaw comprising:
one or more posts slidably disposed in one or more arcuate cam slots of the plurality of arcuate cam slots; and
one or more flexible tabs by which the crimping jaw is snapped into engagement in a slot of the plurality of slots.

2. The prosthetic valve crimping device of claim 1, wherein each slot of the plurality of slots is linear.

3. The prosthetic valve crimping device of claim 1, wherein the one or more posts comprises two posts.

4. The prosthetic valve crimping device of claim 3, wherein each arcuate cam slot of the plurality of arcuate cam slots has a single post of the one or more posts slidably disposed therein.

5. The prosthetic valve crimping device of claim 1, wherein the housing comprises two housing portions, and wherein each housing portion defines multiple arcuate cam slots of the plurality of arcuate cam slots.

6. The prosthetic valve crimping device of claim 1, wherein the housing comprises a handle for a user to rotate the housing relative to the base member.

7. The prosthetic valve crimping device of claim 1, further comprising a rotational limiter pivotably coupled to the base member.

8. The prosthetic valve crimping device of claim 7, wherein the rotational limiter is pivotable between: (i) a first position in which the rotational limiter is spaced apart from the housing and (ii) a second position in which the rotational limiter is engaged with the housing.

9. The prosthetic valve crimping device of claim 1, wherein the housing defines an open center area, wherein an end portion of each crimping jaw is positioned in the open center area, and wherein the end portion of each crimping jaw is wider than the housing.

10. The prosthetic valve crimping device of claim 1, wherein the one or more flexible tabs consists of four flexible tabs.

11. A method of crimping a balloon-expandable prosthetic heart valve onto a balloon of a delivery catheter system, the method comprising:
positioning the balloon-expandable prosthetic heart valve, in an expanded state, over the balloon of the delivery catheter system with the balloon in a deflated state;
radially crimping the balloon-expandable prosthetic heart valve onto the balloon of the delivery catheter system;
positioning a crimping accessory over the radially crimped balloon-expandable prosthetic heart that is on the balloon of the delivery catheter system; and
radially crimping the crimping accessory to further radially compress and form a localized portion of the radially crimped balloon-expandable prosthetic heart that is on the balloon of the delivery catheter system.

12. The method of claim 11, wherein the crimping accessory comprises a compressible portion and at least one incompressible portion.

13. The method of claim 11, wherein the crimping accessory comprises two incompressible portions and a middle compressible portion positioned between the two incompressible portions.

14. The method of claim 13, wherein the crimping accessory further comprises two flared end portions.

15. A prosthetic valve crimping device comprising:
a base member;
a housing that is rotatable relative to the base member, the housing defining a center and a central axis, wherein the housing has a width measured along the central axis; and
a plurality of crimping jaws radially movable relative to the housing, and end portion of each crimping jaw having a width measured along the central axis,
wherein the width of the end portion of each crimping jaw is wider than the width of the housing.

16. The prosthetic valve crimping device of claim 15, further comprising a slotted plate affixed to the base member, the slotted plate defining a plurality of slots extending radially from the center.

17. The prosthetic valve crimping device of claim 16, wherein the plurality of crimping jaws are slidably disposed in the plurality of slots.

18. The prosthetic valve crimping device of claim 15, wherein the housing comprises two housing portions, and wherein each housing portion defines multiple arcuate cam slots of the plurality of arcuate cam slots.

19. The prosthetic valve crimping device of claim 18, wherein each crimping jaw comprises two posts.

20. The prosthetic valve crimping device of claim 19, wherein each arcuate cam slot of the plurality of arcuate cam slots has a single post of the plurality of crimping jaws slidably disposed therein.
